(19) United States
(12) Patent Application Publication
(10) Pub. No.: US 2024/0238313 A1
(43) Pub. Date: Jul. 18, 2024

(54) DOSING OF CV-10155 IN THE EVENING OR PRIOR TO SLEEP TO TREAT GABAA DISORDERS

(71) Applicant: Eliem Therapeutics (UK) Ltd, Altrincham (GB)

(72) Inventors: Joanne Palmer-Phillips, Boston, MA (US); Elizabeth Mitchell, Boston, MA (US); Valerie Morisset, Boston, MA (US); Amy Chappell, Boston, MA (US)

(21) Appl. No.: 18/225,930

(22) Filed: Jul. 25, 2023

Related U.S. Application Data
(63) Continuation of application No. 17/698,286, filed on Mar. 18, 2022, now abandoned.
(60) Provisional application No. 63/163,253, filed on Mar. 19, 2021.

(51) Int. Cl. A61K 31/58; A61P 25/08; A61P 25/22; A61P 25/24

(52) U.S. Cl. CPC A61K 31/58; A61P 25/08; A61P 25/22; A61P 25/24

(57) ABSTRACT

The invention provides methods for treating a GABA_{A} disorders by providing a composition containing CV-10155 to a subject in the evening or prior to a period of sleep by the subject.

### FIELD OF THE INVENTION

The invention relates generally to methods for treating disorders of the central nervous systems (CNS) associated with altered activity of GABA_{A} receptors.

### BACKGROUND

According the World Health Organization (WHO), neurological disorders affect up to one billion people worldwide. Neurological disorders include a wide range of conditions, such as Alzheimer's disease, brain injuries, epilepsy, headache, infections, multiple sclerosis, and Parkinson's disease, and stroke. Many neurological disorders stem from altered signaling by receptors for the neurotransmitter γ-aminobutyric acid (GABA). GABA_{A} recseptors are pentameric transmembrane receptors that include various combinations of 19 different subunit polypeptides. At least 15 GABA_{A} receptor subtypes are known, and particular subtypes are associated with different conditions. For example, altered activity of receptor subtypes that include α₂ or α₃ subunits is associated with anxiety disorders, whereas α₅-containing subtypes appear to play a role in memory and cognition.

Neuroactive steroids that alter the activity of GABA_{A} receptors have been investigated as drug candidates for a variety of neurological disorders. One neurosteroid being investigated as a therapeutic candidate is CV-10155, which has the IUPAC name 1-[2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-oxoethyl]pyrazole-4-carbonitrile. Compositions containing isomerically pure CV-10155 preferentially potentiate α4β3δ GABA_{A} receptors as compared to α1β2γ2 GABA_{A} receptors and thus are useful to treat GABA_{A} disorders, such as anxiety, depression, and seizure disorders, with a minimum of side effects.

### SUMMARY

The invention provides methods for treating a GABA_{A} disorder by providing a composition containing CV-10155 to a subject at a time when the subject is preparing to sleep, such as in the evening. The invention is based on the discovery that administration of CV-10155 promotes sleep and improves sleep quality. Thus, the methods of the invention are useful disorders of the central nervous system (CNS) that are associated with sleep disturbances.

In an aspect, the invention provides methods for treating a GABA_{A} disorder in a subject by providing to the subject a composition comprising a compound of Formula (I):

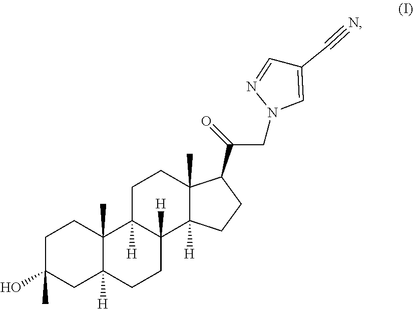

in the evening of the subject's local time. The compound of Formula (I) is also referred to herein as CV-10155.

The evening may be defined by any suitable measure. For example, the evening may be the part of the day that starts 3 p.m., 4 p.m., 5 p.m., 6 p.m., 7 p.m., 8 p.m., or 9 p.m. of the subject's local time. The evening may be determined in relation to the local time of the sunset. For example, the evening may commence at sunset, at 15 minutes before sunset, at 30 minutes before sunset, at 45 minutes before sunset, at 60 minutes before sunset, at 90 minutes before sunset, at 2 hours before sunset, at 2.5 hours before sunset, at 3 hours before sunset, at 3.5 hours before sunset, at 4 hours before sunset, at 15 minutes after sunset, at 30 minutes after sunset, at 45 minutes after sunset, at 60 minutes after sunset, at 90 minutes after sunset, at 2 hours after sunset, at 2.5 hours after sunset, at 3 hours after sunset, at 3.5 hours after sunset, or at 4 hours after sunset. The evening may be defined relation to when the subject goes to sleep, For example, the evening may commence when the subject goes to sleep, 0.5 hours before the subject goes to sleep, 1 hour before the subject goes to sleep, 1.5 hours before the subject goes to sleep, 2 hours before the subject goes to sleep, 2.5 hours before the subject goes to sleep, 3 hours before the subject goes to sleep, 3.5 hours before the subject goes to sleep, 4 hour before the subject goes to sleep, 4.5 hours before the subject goes to sleep, 5 hours before the subject goes to sleep, 5.5 hours before the subject goes to sleep, or 6 hours before the subject goes to sleep.

The composition may be chemically pure, i.e., free from other molecules or chemical species. For example, the other molecule or chemical species may have a distinct chemical formula, structural formula, empirical formula, molecular formula, or condensed formula. The composition may have a defined level of chemical purity. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of a mixture that includes the compound of Formula (I) and one or more distinct molecules or chemical species.

The composition may be isomerically pure with respect to all isomers. The composition may be isomerically pure with respect to one or more particular types of isomers. The composition may be substantially free of structural isomers or a particular type of structural isomers, such as a regioisomers. The composition may be substantially free of stereoisomers or a particular type of stereoisomers, such as enantiomers or diastereomers.

The composition may contain the compound of Formula (I) at any level of isomeric purity to achieve preferential modulation of an α4β3δ GABA_{A} receptor as compared to an α1β2γ2 GABA_{A} receptor. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may contain the compound of Formula (I) and be substantially free of stereoisomers. The stereoisomer may differ from Formula (I) at one, two, three, four, five, six, seven, or eight chiral centers. The stereoisomer may be a diastereomer or an enantiomer. For example, the stereoisomer may be a compound of Formulas (II) or (III):

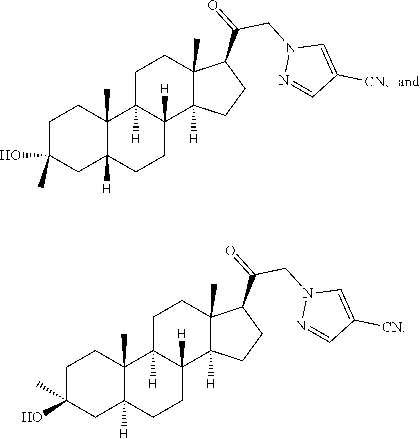

The composition may contain one or more stereoisomers of the compound of Formula (I), such as a compound of Formula (II) or (III), at less than 5%, less than 4%, less than 3%, less than 2%, less than 1%, less than 0.5%, or less than 0.1% of the total of the compound of Formula (I) and the one or more stereoisomers thereof. The composition may contain the compound of Formula (I) and one or more stereoisomer thereof at a ratio of at least 19:1, 20:1, 25:1, 30:1, 40:1, 50:1, 100:1, 200:1, 500:1, or 1000:1.

The compound may potentiate a GABA_{A} receptor, a GABA_{A} receptor subtype, or a subset of GABA_{A} receptor subtypes by any mechanism. The compound may potentiate a GABA_{A} receptor, subtype, or subset by allosteric modulation, activation, or inhibition. The allosteric modulation may be positive or negative.

The composition may preferentially potentiate an α4β3δ GABA_{A} receptor as compared to an α1β2γ2 GABA_{A} receptor to any degree. The composition may preferentially potentiate an α4β3δ GABA_{A} receptor as compared to an α1β2γ2 GABA_{A} receptor by any measure or parameter.

The composition may have an EC₅₀ for α4β3δ GABA_{A} receptors that is lower than the EC₅₀ for α1β2γ2 GABA_{A} receptors. The EC₅₀ for α4β3δ GABA_{A} receptors may be lower than the EC₅₀ for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold. The EC₅₀ for α4β3δ GABA_{A} receptors may be less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the EC₅₀ for α1β2γ2 GABA_{A} receptors.

The composition may have a binding affinity (which may be expressed, e.g., as a dissociation constant K_{D}) for α4β3δ GABA_{A} receptors that is lower than the binding affinity for α1β2γ2 GABA_{A} receptors. The binding affinity for α4β3δ GABA_{A} receptors may be lower than the binding affinity for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold. The binding affinity for α4β3δ GABA_{A} receptors may be less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the binding affinity for α1β2γ2 GABA_{A} receptors.

The composition may have an EC₅₀ for α4β3δ GABA_{A} receptors that is below a defined value. The composition may have an EC₅₀ for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

The composition may have a binding affinity for α4β3δ GABA_{A} receptors that is below a defined value. The composition may have an binding affinity for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

The method may be useful for treating any GABA_{A} disorder. The GABA_{A} disorder may be any disease, disorder, or condition associated with altered GABA_{A} receptor function or any disorder may be disease, disorder, or condition that can be ameliorated by altered GABA_{A} receptor function. The GABA_{A} disorder may be acute pain, an addictive disorder, Alzheimer's disease, Angelman's syndrome, anti-social personality disorder, an anxiety disorder, attention deficit hyperactivity disorder (ADHD), an attention disorder, an auditory disorder, autism, an autism spectrum disorder, bipolar disorder, chronic pain, a cognitive disorder, a compulsive disorder, a convulsive disorder, dementia, depression, dysthymia, an epileptic disorder, essential tremor, epileptogenesis, fragile X syndrome, generalized anxiety disorder (GAD), Huntington's disease, injury related pain syndrome, insomnia, ischemia, Lewis body type dementia, a memory disorder, migraines, a mood disorder, movement disorder, a neurodegenerative disease, neuropathic pain, an obsessive compulsive disorder, pain, a panic disorder, Parkinson's disease, a personality disorder, posttraumatic stress disorder (PTSD), psychosis, Rett syndrome, a schizoaffective disorder, schizophrenia, a schizophrenia spectrum disorder, a seizure disorder, a sleep disorder, social anxiety disorder, status epilepticus, stress, stroke, tinnitus, traumatic brain injury (TBI), vascular disease, vascular malformation, vascular type dementia movement disorder, Wilson's disease, or withdrawal syndrome.

Providing may include administering the composition to the subject. The composition may be administered by any suitable means or route, such as orally, intravenously, enterally, parenterally, dermally, buccally, topically (including transdermally), by injection, nasally, pulmonarily, and with or on an implantable medical device (e.g., stent or drug-eluting stent or balloon equivalents).

The composition may be provided as a single unit dosage. The composition may be provided as a divided dosage.

The composition may be provided according to a dosing regimen that extends for a defined period. For example, multiple doses may be provided over a period of 1 day, 2 days, 3 days, 4 days, 5 days, 1 week, 2 weeks, 3 weeks, 4 weeks, or more. The compositions may be provided repeatedly for a specified duration. For example and without limitation, the compositions may be provided for 1 week, 2 weeks, 3 weeks, 4 weeks, 6 weeks, 8 weeks, 12 weeks, 3 months, 4 months, 5 months, 6 months, 8 months, 10 months, 12 months or more.

In another aspect, the invention provides methods for treating a GABA_{A} disorder in a subject by providing to the subject a composition comprising a compound of Formula (I):

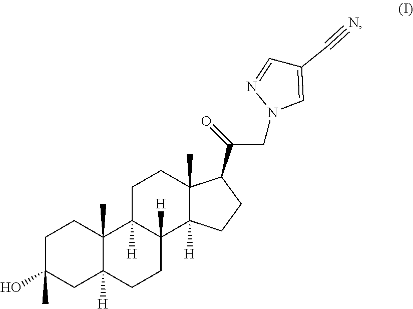

at a dosing time that is less than 6 hours before a time when the subject goes to sleep.

The composition may be provided to the subject less than 5 hours before a time when the subject goes to sleep, less than 4 hours before a time when the subject goes to sleep, less than 3 hours before a time when the subject goes to sleep, less than 2 hours before a time when the subject goes to sleep, or less than 1 hour before a time when the subject goes to sleep.

The composition may be chemically pure and/or isomerically pure. The composition may have a purity in accordance with any standard described above.

The compound may potentiate a GABA_{A} receptor, a GABA_{A} receptor subtype, or a subset of GABA_{A} receptor subtypes by any mechanism. The compound may potentiate a GABA_{A} receptor, subtype, or subset by allosteric modulation, activation, or inhibition. The allosteric modulation may be positive or negative.

The composition may preferentially potentiate an α4β3δ GABA_{A} receptor as compared to an α1β2γ2 GABA_{A} receptor to any degree or by any measure described above.

The method may be useful for treating any GABA_{A} disorder, such as any of those described above.

The composition may be provided by an suitable means or route of administration, such as any of those described above.

The composition may be provided as a single unit dosage or as a divided dosage.

### BRIEF DESCRIPTION OF THE DRAWINGS

FIG. 1 is a graph of average plasma concentration of CV-10155 at various time points following oral administration to dogs.

FIG. 2 is a graph of average plasma concentration of CV-10155 at various time points following oral administration to dogs.

FIG. 3 is a graph of plasma concentration of CV-10155 in individual dogs at various time points following oral administration of 1 mg/kg CV-10155 in a formulation containing labrasol.

FIG. 4 is a graph of plasma concentration of CV-10155 in individual dogs at various time points following oral administration of 1 mg/kg CV-10155 in a formulation containing labrasol/capryol 80:20.

FIG. 5 is a graph of plasma concentration of CV-10155 in individual dogs at various time points following oral administration of 1 mg/kg CV-10155 in a formulation containing 30% 2-hydroxypropyl-beta-cyclodextrin (HPbCD).

FIG. 6 is a graph of plasma concentration of CV-10155 in individual dogs at various time points following intravenous administration of 1 mg/kg CV-10155 in a formulation containing 30% HPbCD.

FIG. 7 is a graph of plasma concentration of CV-10155 in individual dogs at various time points following oral administration of 1 mg/kg CV-10155 in a formulation containing 1:4 HPMC-AS-MG.

FIG. 8 is a graph of plasma concentration of CV-10155 in individual dogs at various time points following oral administration of 1 mg/kg CV-10155 in a formulation containing 1:4 HPMC-E3.

FIG. 9 is a graph of plasma concentration of CV-10155 in individual dogs at various time points following oral administration of 1 mg/kg CV-10155 in a formulation containing 1:4 PVP VA64.

FIG. 10 is a graph of plasma concentration of CV-10155 in individual dogs at various time points following oral administration of 1 mg/kg CV-10155 in a formulation containing 1:4 Eudragit L100-55.

FIG. 11 is a graph of the average plasma concentration of CV-10155 following oral administration to humans.

FIG. 12 is a graph of the average plasma concentration of CV-10155 following oral administration of a 30 mg dose to humans.

FIG. 13 is a graph showing the ratios of Cₘₐₓ and AUC between fed and fasted subjects.

FIG. 14 is a graph of the average plasma concentration of CV-10155 following oral administration to humans.

FIG. 15 is a graph of Cₘₐₓ ranges from studies on rats, dogs, and humans.

FIG. 16 is a graph of AUC₀₋₂₄ ranges from studies on rats, dogs, and humans. B

FIG. 17 is a hypnogram showing the percentage of time spent in different sleep states by rats given drug vehicle.

FIG. 18 is a hypnogram showing the percentage of time spent in different sleep states by rats given 1 mg/kg CV-10155.

FIG. 19 is a hypnogram showing the percentage of time spent in different sleep states by rats given 3 mg/kg CV-10155.

FIG. 20 is a hypnogram showing the percentage of time spent in different sleep states by rats given 6 mg/kg CV-10155.

FIG. 21 is a hypnogram showing the percentage of time spent in different sleep states by rats given 10 mg/kg CV-10155.

### DETAILED DESCRIPTION

### Definitions

As used herein, a “pure isomeric” compound or “isomerically pure” compound is substantially free of other isomers of the compound. The term “pure isomeric” compound or “isomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified structure. In certain embodiments, the weights are based upon total weight of all isomers of the compound.

As used herein, a “pure stereoisomeric” compound or “stereoisomerically pure” compound is substantially free of other stereoisomers of the compound. Thus, the composition is substantially free of isomers that differ at any chiral center. If the compound has multiple chiral centers, a substantial majority of the composition contains compounds having identical stereochemistry at all of the chiral centers. The term “pure stereoisomeric” compound or “stereoisomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified stereochemistry. In certain embodiments, the weights are based upon total weight of all stereoisomers of the compound.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the enantiomer. In certain embodiments, the weights are based upon total weight of all enantiomers or stereoisomers of the compound.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; N may be any isotopic form, including ¹⁴N and ¹⁵N; O may be in any isotopic form, including ¹⁶O and ¹⁸O; and the like.

The articles “a” and “an” may be used herein to refer to one or to more than one (i.e. at least one) of the grammatical objects of the article. By way of example “an analogue” means one analogue or more than one analogue.

As used herein, the terms “modulation” and “potentiation” refer to the inhibition or stimulation of GABA receptor function. A “modulator” or “potentiator” may be, for example, an agonist, partial agonist, antagonist, or partial antagonist of the GABA receptor. The “modulator” or “potentiator” may act at the active site or at an allosteric site on a GABA receptor. It may promote or inhibit ligand binding. It may facilitate or attenuate ligand-mediated, e.g., GABA-mediated, signaling.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, el al., J. Pharm. Sci. (1977) 66(1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates, and methanolates.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³N, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers”, and those that are non-superimposable mirror images of each other are termed “enantiomers.” When a compound has an asymmetric center, for example, and an atom, such as a carbon atom, is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, a human (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder, or condition, or one or more symptoms associated with the disease, disorder, or condition, or prevent its recurrence. A prophylactically effective amount of a compound means an amount of a therapeutic agent, alone or in combination with other agents, which provides a prophylactic benefit in the prevention of the disease, disorder, or condition. The term “prophylactically effective amount” can encompass an amount that improves overall prophylaxis or enhances the prophylactic efficacy of another prophylactic agent.

Providing Doses of CV-10155 Prior to Sleep and/or in the Evening

The invention is based on the discovery that administration of CV-10155, which is useful for treating a variety of central nervous system (CNS) disorders associated with altered activity of GABA_{A} receptors, promotes sleep and improves sleep quality. Therefore, methods of the invention include providing a dose of CV-10155 prior to a period of sleep and/or in the evening. Consequently, methods of the invention are useful disorders of the central nervous system (CNS) that are associated with sleep disturbances.

Methods may include providing a dose of a composition that contains CV-10155 to a subject in the evening. The term “evening” refers to a period of the day in the local time zone of the subject.

The evening may be defined by any suitable measure. For example, the evening may be the part of the day that starts 3 p.m., 4 p.m., 5 p.m., 6 p.m., 7 p.m., 8 p.m., or 9 p.m. of the subject's local time. The evening may be determined in relation to the local time of the sunset. For example, the evening may commence at sunset, at 15 minutes before sunset, at 30 minutes before sunset, at 45 minutes before sunset, at 60 minutes before sunset, at 90 minutes before sunset, at 2 hours before sunset, at 2.5 hours before sunset, at 3 hours before sunset, at 3.5 hours before sunset, at 4 hours before sunset, at 15 minutes after sunset, at 30 minutes after sunset, at 45 minutes after sunset, at 60 minutes after sunset, at 90 minutes after sunset, at 2 hours after sunset, at 2.5 hours after sunset, at 3 hours after sunset, at 3.5 hours after sunset, or at 4 hours after sunset. The evening may be defined relation to when the subject goes to sleep, For example, the evening may commence when the subject goes to sleep, 0.5 hours before the subject goes to sleep, 1 hour before the subject goes to sleep, 1.5 hours before the subject goes to sleep, 2 hours before the subject goes to sleep, 2.5 hours before the subject goes to sleep, 3 hours before the subject goes to sleep, 3.5 hours before the subject goes to sleep, 4 hour before the subject goes to sleep, 4.5 hours before the subject goes to sleep, 5 hours before the subject goes to sleep, 5.5 hours before the subject goes to sleep, or 6 hours before the subject goes to sleep.

Alternatively or additionally, methods may include providing a dose of a composition that contains CV-10155 to a subject prior to a period of sleep by the subject. The period of sleep may be an extended period, such as the duration for which individuals typically sleep during the night. For example and without limitation, the duration of the period of sleep may be at least 3 hours, at least 4 hours, at least 5 hours, at least 6 hours, at least 7 hours, at least 8 hours, at least 9 hours, at least 10 hours. The period of sleep may be a nap, or it may be distinct from a nap. The period of sleep may occur partially or completely during nightfall. Alternatively, the period of sleep may occur during daylight.

The dose may be provided within a time interval prior to a period of sleep by the subject. For example and without limitation, the composition may be provided to the subject less than 8 hours before a time when the subject goes to sleep, less than 7 hours before a time when the subject goes to sleep, less than 6 hours before a time when the subject goes to sleep, less than 5 hours before a time when the subject goes to sleep, less than 4 hours before a time when the subject goes to sleep, less than 3 hours before a time when the subject goes to sleep, less than 2 hours before a time when the subject goes to sleep, or less than 1 hour before a time when the subject goes to sleep.

### Compositions

Methods of the invention include compositions that contain the compound of Formula (I):

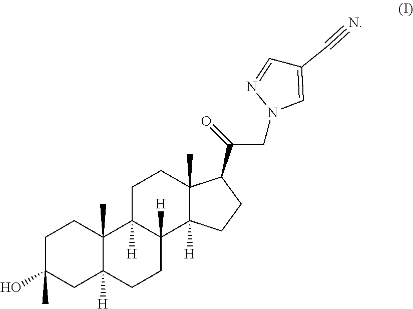

The compound of Formula (I), which is referred to herein as CV-10155, is known in the art and described in, for example, U.S. Pat. No. 10,857,163 and International Patent Publication No. WO 2016/061527.

The composition may be chemically pure, i.e., free from other molecules or chemical species. For example, the other molecule or chemical species may have a distinct chemical formula, structural formula, empirical formula, molecular formula, or condensed formula. The composition may have a defined level of chemical purity. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of a mixture that includes the compound of Formula (I) and one or more distinct molecules or chemical species.

The composition may be isomerically pure. The composition may contain the compound of Formula (I) at any level of isomeric purity, i.e., the composition may contain the compound of Formula (I) at a level in relation to an isomeric form of the compound. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may be isomerically pure with respect to all isomers. The composition may be isomerically pure with respect to one or more particular types of isomers. The composition may be substantially free of structural isomers or a particular type of structural isomers, such as a regioisomers. The composition may be substantially free of stereoisomers or a particular type of stereoisomers, such as enantiomers or diastereomers.

The composition may contain the compound of Formula (I) at any level of isomeric purity to achieve preferential modulation of an α4β3δ GABA_{A} receptor as compared to an α1β2γ2 GABA_{A} receptor. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof. The importance is isomeric purity of compositions containing the compound of Formula (I) to achieve preferential modulation of an α4β3δ GABA_{A} receptor as compared to an α1β2γ2 GABA_{A} receptor is described in U.S. Pat. No. 10,857,163.

The composition may contain the compound of Formula (I) and be substantially free of stereoisomers. The stereoisomer may differ from Formula (I) at one, two, three, four, five, six, seven, or eight chiral centers. The stereoisomer may be a diastereomer or an enantiomer. For example, the stereoisomer may be a compound of Formulas (II) or (III):

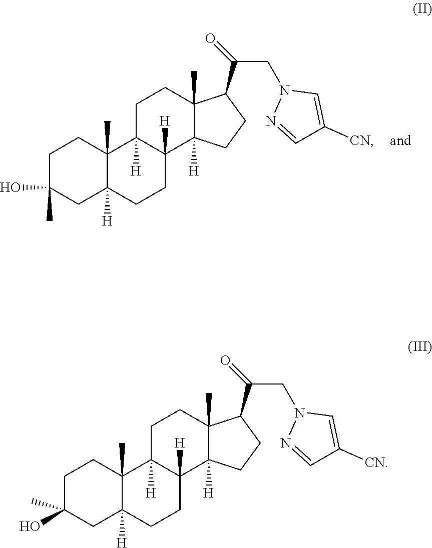

The composition may contain one or more stereoisomers of the compound of Formula (I), such as a compound of Formula (II) or (III), at less than 5%, less than 4%, less than 3%, less than 2%, less than 1%, less than 0.5%, or less than 0.1% of the total of the compound of Formula (I) and the one or more stereoisomers thereof. The composition may contain the compound of Formula (I) and one or more stereoisomer thereof at a ratio of at least 19:1, 20:1, 25:1, 30:1, 40:1, 50:1, 100:1, 200:1, 500:1, or 1000:1.

A pharmaceutical composition containing the compound of Formula (I) may be in a form suitable for oral use, such as tablets, troches, lozenges, fast-melts, aqueous or oily suspensions, dispersible powders or granules, emulsions, hard or soft capsules, syrups, or elixirs. Compositions intended for oral use may be prepared according to any method known in the art for the manufacture of pharmaceutical compositions and such compositions may contain one or more agents selected from sweetening agents, flavoring agents, coloring agents, and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid, or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452; and 4,265,874, the contents of which are incorporated herein by reference, to form osmotic therapeutic tablets for control release. Preparation and administration of compounds is discussed in U.S. Pat. No. 6,214,841 and U.S. Pub. No. 2003/0232877, the contents of which are incorporated herein by reference.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin, or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring, and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

In certain embodiments, the formulation is not a sustained release formulation. In certain embodiments, the formulation is not injectable. In certain embodiments, the formulation does not contain particles having a D50 (volume weighted median diameter) of less than 10 microns. In certain embodiments, the formulation does not contain a polymer surface stabilizer. In certain embodiments, the formulation is not an aqueous suspension.

The composition may be formulated for administration by a particular mechanism. The composition may be formulated for oral, intravenous, enteral, parenteral, dermal, buccal, topical nasal, or pulmonary administration. The composition may be formulated for administration by injection or on an implantable medical device (e.g., stent or drug-eluting stent or balloon equivalents).

The composition may be formulated a single daily dosage. The composition may be formulated for multiple daily doses, e.g., two, three, four, five, six or more daily doses.

The composition may be provided to the subject according to any dosing schedule. The composition may be provided once per day. The composition may be provided multiple times per day.

### Treatment of GABA_{A} Receptor Disorders

The methods of the invention are useful for treating disorders that are associated with, or can be ameliorated by, alteration of activity of a GABA_{A} receptor. GABA_{A} receptors are ligand-gated ion channels that selectively allow Cl⁻ ions to pass through the plasma membrane upon binding of GABA. GABA_{A} receptors are expressed in neurons throughout the central nervous system (CNS) and mediate most of the physiological activities of GABA in the CNS. Within neurons, the type and density of GABA_{A} receptors can vary between cell bodies and dendrites. GABA_{A} receptors are also expressed in other tissues, including Leydig cells, placenta, immune cells, liver, bone growth plates, and other endocrine tissues. Outside the CNS, GABA_{A} receptors can regulate cell proliferation and immune responses.

Structurally, GABA_{A} receptors are pentamers that include five polypeptide subunits. The polypeptide subunits are encoded by 19 genes that are grouped as follows based on sequence similarity: α(1-6), β(1-3), γ(1-3), δ, ε, θ, π, and ρ(1-3). Most subtypes are heteropentamers that include two copies of one type of α subunit, two copies of one type of β subunit, and one copy of one type of γ, δ, ε, θ, or π subunit; other subtypes are homopentamers or heteropentamers of ρ subunits. Known subtypes of GABA_{A} receptors include α1β1γ2, α1β2γ2, α1β3γ2, α2β1γ2, α2β2γ2, α2β3γ2, α3β1γ2, α3β2γ2, α3β3γ2, α4β1γ2, α4β3δ, α4β3γ2, α5β1γ2, α5β2γ2, α5β3γ2, α6β1γ2, α6β2γ2, GABA_{A} receptor subtypes vary among tissue types and anatomical regions of the CNS, and subtypes may be associated with specific functions. In addition, GABA_{A} receptor subtypes may vary between normal and malignant cells of the same tissue type.

The active site of a GABA_{A} receptor is the binding site for GABA and for drugs such as muscimol, gaboxadol, and bicuculline. GABA_{A} receptors also have several allosteric binding sites that are the targets of other drugs, including benzodiazepines, nonbenzodiazepines, neuroactive steroids, barbiturates, ethanol, inhaled anaesthetics, and picrotoxin. Thus, the activity of GABA_{A} receptors is controlled by binding of molecules to both the active and allosteric binding sites. The structure, function, and regulation of GABA_{A} receptors are known in the art and described in, for example, Sigel E., and Steinmann, M.E., Structure, Function, and Modulation of GABA_{A} Receptors, J. Biol. Chem. 287:48 pp. 40224-402311 (2012), doi: 10.1074/jbc.R112.386664, the contents of which are incorporated herein by reference.

The isomerically pure compositions used in methods of the invention preferentially potentiate the activity selected GABA_{A} receptor subtypes. The compositions may preferentially potentiate the activity of one or more GABA_{A} receptor subtypes, such as those described above, relative to one or more GABA_{A} receptor subtypes. In certain embodiments, the compositions preferentially potentiate the activity of α4β3δ receptors compared to α1β2γ2 receptors. The ability of isomerically pure forms of the compound of Formula (I) to preferentially modulate α4β3δ GABA_{A} receptors as compared to α1β2γ2 GABA_{A} receptors is described in U.S. Pat. No. 10,857,163.

The compositions used in methods of the invention may potentiate one or more GABA_{A} receptors by any mechanism. For example, and without limitation, the isomerically pure form of CV-10155 may potentiate a GABA_{A} receptor by allosteric modulation, activation, or inhibition. The allosteric modulation may be positive or negative.

The preferential activity of a composition on one or more GABA_{A} receptor as compared to one or more other GABA_{A} receptor may be measured by any suitable means. Activity may be measure using in vitro assays or in vivo assays. For example and without limitation, methods of measuring the effect of modulators on GABA_{A} receptor activity include anticonvulsant assays, binding assays, fluorescence membrane potential assays, immune response assays, intracranial self-stimulation assays patch clamps assays, proliferation assays receptor occupancy assays seizure induction assays, e.g., using pentylenetetrazol (PTZ) or maximal electroshock (MES), and survival assays. Such assays are known in the art and described in, for example, International Publication No. WO 2016/061527; Ghisdal P., et al., Determining the relative efficacy of positive allosteric modulators of the GABA_{A} receptor: design of a screening approach, J Biomol Screen. 2014 Mar; 19(3):462-7. doi: 10.1177/1087057113501555, Epub 2013 Aug 29; Tian J., et al., Clinically applicable GABA receptor positive allosteric modulators promote β-cell replication, Sci Rep. 2017 Mar 23;7(1):374. doi: 10.1038/s41598-017-00515-y; and Tian J., et al., A Clinically Applicable Positive Allosteric Modulator of GABA Receptors Promotes Human B-Cell Replication and Survival as well as GABA's Ability to Inhibit Inflammatory T Cells, J Diabetes Res. 2019 Feb 26;2019:5783545, doi: 10.1155/2019/5783545, the contents of each of which are incorporated herein by reference.

The preferential activity of a composition on one or more GABA_{A} receptors as compared to one or more other GABA_{A} receptors may be expressed by any suitable means. For example and without limitation, the preferential activity may be indicated by a comparison of EC₅₀ values or binding affinity values.

In certain embodiments, compositions used in methods of the invention have an EC50 for α4β3δ GABA_{A} receptors that is lower than the EC₅₀ for α1β2γ2 GABA_{A} receptors. The EC₅₀ for α4β3δ GABA_{A} receptors may be lower than the EC₅₀ for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions used in methods of the invention have an EC₅₀ for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the EC50 for α1β2γ2 GABA_{A} receptors.

In certain embodiments, compositions used in methods of the invention have an binding affinity (which may be expressed, e.g., as a dissociation constant K_{D}) for α4β3δ GABA_{A} receptors that is lower than the binding affinity for α1β2γ2 GABA_{A} receptors. The binding affinity for α4β3δ GABA_{A} receptors may be lower than the binding affinity for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions used in methods of the invention have an binding affinity for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the binding affinity for α1β2γ2 GABA_{A} receptors.

In certain embodiments, compositions used in methods of the invention have an EC50 for α4β3δ GABA_{A} receptors that is below a defined value. For example and without limitation, the composition may have an EC50 for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

In certain embodiments, compositions used in methods of the invention have an binding affinity for α4β3δ GABA_{A} receptors below a defined value. For example and without limitation, the composition may have an binding affinity for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

The methods of the invention may be effective for treatment of a GABA_{A} disorder. The GABA_{A} disorder may be any disease, disorder, or condition associated with altered GABA_{A} receptor function or any disorder may be disease, disorder, or condition that can be ameliorated by altered GABA_{A} receptor function. The GABA_{A} disorder may be acute pain, an addictive disorder, Alzheimer's disease, Angelman's syndrome, anti-social personality disorder, an anxiety disorder, attention deficit hyperactivity disorder (ADHD), an attention disorder, an auditory disorder, autism, an autism spectrum disorder, bipolar disorder, chronic pain, a cognitive disorder, a compulsive disorder, a convulsive disorder, dementia, depression, dysthymia, an epileptic disorder, essential tremor, epileptogenesis, fragile X syndrome, generalized anxiety disorder (GAD), Huntington's disease, injury related pain syndrome, insomnia, ischemia, Lewis body type dementia, a memory disorder, migraines, a mood disorder, movement disorder, a neurodegenerative disease, neuropathic pain, an obsessive compulsive disorder, pain, a panic disorder, Parkinson's disease, a personality disorder, posttraumatic stress disorder (PTSD), psychosis, Rett syndrome, a schizoaffective disorder, schizophrenia, a schizophrenia spectrum disorder, a seizure disorder, a sleep disorder, social anxiety disorder, status epilepticus, stress, stroke, tinnitus, traumatic brain injury (TBI), vascular disease, vascular malformation, vascular type dementia movement disorder, Wilson's disease, or withdrawal syndrome.

The methods of treating a subject include providing a composition of the invention, as described above, to the subject. Providing may include administering the composition to the subject. The composition may be administered by any suitable route or means, such as orally, intravenously, enterally, parenterally, dermally, buccally, topically (including transdermally), by injection, nasally, pulmonarily, and with or on an implantable medical device (e.g., stent or drug-eluting stent or balloon equivalents).

The composition may be provided as a single unit dosage. The composition may be provided as a divided dosage.

The composition may be provided under any suitable dosing regimen. For example, the composition may be provided as a single dose or in multiple doses. Multiple doses may be provided in provided separated by intervals, such as 12 hours, 24 hours, 2 days, 3 days, 4 days, 5 days, 1 week, 2 weeks, 3 weeks, 4 weeks, or more. Multiple doses may be provided within a period of time. For example, multiple doses may be provided over a period of 1 day, 2 days, 3 days, 4 days, 5 days, 1 week, 2 weeks, 3 weeks, 4 weeks, or more. The compositions may be provided repeatedly for a specified duration. For example and without limitation, the compositions may be provided for 1 week, 2 weeks, 3 weeks, 4 weeks, 6 weeks, 8 weeks, 12 weeks, 3 months, 4 months, 5 months, 6 months, 8 months, 10 months, 12 months or more.

### EXAMPLES

Aspects of the invention are illustrated in the examples provided below. In the examples and accompanying figures, the compound of Formula (I) is alternately referred to as CV-10155, ATN-10155, ATX-10155, ATH-155, CTP-10155, ETX-155

### Example 1

The pharmacokinetics of CV-10155 were analyzed in beagle dogs. Oral formulations of CV-10155 included cyclodextrin solutions; lipid solutions in softgel capsules; and amorphous, solid, spray-dried dispersions (SDD).

Sixteen male beagles weighing between 9.9 kg and 12 kg were administered a solid dosage form containing either hydroxypropyl methylcellulose acetate succinate (HPMC-AS) or poly(1-vinylpyrrolidone-co-vinyl acetate) (PVP-VA64. Doses were administered to dogs in either fed or fasted state, and pentagastrin was administered prior to CV-10155 dose in dogs that were fasted post-dose. Following doses, dogs received water via syringe and gavage tube at 5 mL/kg. Dosing regimens are summarized in Table 1.

TABLE 1

| Formulation | Dose (mg) | Pentagastrin* | fed/fasted |
|---|---|---|---|
| 1:4 HPMC-AS | 10 | Yes | 4 hr post-dose |
| 1:4 HPMC-AS | 10 | No | 1 hr pre-dose |
| 1:4 HPMC-AS | 30 | Yes | 4 hr post-dose |
| 1:4 PVP-VA64 | 10 | Yes | 4 hr post-dose |

*Pentagastrin doses (6 μg/kg, 0.24 μg/mL, 0.25 mL/kg) were administered by intramuscular injection 30 minutes prior to CV-10155 capsule dose.

Pharmacokinetic parameters of CV-10155 in dogs is summarized in Table 2.

TABLE 2

| | Formulation (10 mg tablet/dog) | | | |
|---|---|---|---|---|
| PK Parameter* | 1:4 HPMC-AS | 1:4 PVP-VA64 | HPMC-AS | HPMC-AS** |
| Cₘₐₓ (ng/ml) | 241 | 122 | 442 | 922 |
| Tₘₐₓ (hr) | 1.50 | 2.50 | 1.13 | 1.00 |
| T_{1/2} (hr) | 13.8 | 10.4 | 13.1 | 13.4 |
| F (%)*** | 34.7 | 19.8 | 47.8 | 38.6 |

*Mean of 4 animals/group
**30 mg/dog (three 10 mg tablets)
***Based on mean AUC (dose-normalized) from 4 dogs receiving 1.0 mg/kg IV dose
CL = 2.17 ml/min/kg
Vdss = 1.92 L/kg
T_{1/2} = 13.9 hr

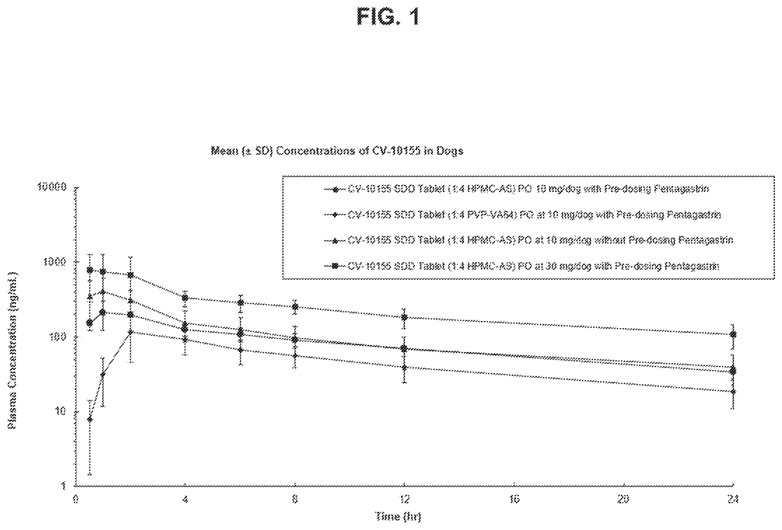

FIG. 1 is a graph of average plasma concentration of CV-10155 at various time points following oral administration to dogs.

Raw data from the pentagastrin/fasted cohort that received 10 mg CV-10155 in HPMC-AS-formulation are shown in Table 3; raw data from the fed cohort that received 10 mg CV-10155 in HPMC-AS-formulation are shown in Table 4; and raw data from the pentagastrin/fasted cohort that received 30 mg CV-10155 in HPMC-AS-formulation are shown in Table 5.

TABLE 3

| | No. of | Body | Actual | | | | | | | | AUC_{Inf}/D | |
|---|---|---|---|---|---|---|---|---|---|---|---|---|
| | pts used | Weight | Dosage | T_{1/2} | Tₘₐₓ | Cₘₐₓ | AUCₗₐₛₜ | AUC_{Inf} | AUC_{Extr} | MRT_{Inf} | (hr * kg * ng/ | F |
| Animal | for T_{1/2} | (kg) | (mg/kg) | (hr) | (hr) | (ng/mL) | (hr * ng/mL) | (hr * ng/mL) | (%) | (hr) | mL/mg) | (%) |
| Dog #1 | 3 | 10.0 | 1.00 | 25.7 | 2 | 183 | 1754 | 3314 | 47.1 | 33.6 | 3314 | 29.1 |
| (8997722) | | | | | | | | | | | | |
| Dog #2 | 3 | 10.8 | 0.926 | 9.66 | 1 | 217 | 2145 | 2612 | 17.9 | 13.6 | 2821 | 38.5 |
| (8229203) | | | | | | | | | | | | |
| Dog #3 | 3 | 10.5 | 0.952 | 11.1 | 2 | 222 | 2093 | 2630 | 20.4 | 14.8 | 2762 | 36.5 |
| (8235203) | | | | | | | | | | | | |
| Dog #4 | 3 | 9.90 | 1.01 | 8.84 | 1 | 340 | 2118 | 2465 | 14.1 | 11.6 | 2441 | 34.8 |
| (8229972) | | | | | | | | | | | | |
| Mean | | 10.3 | 0.97 | 13.8 | 1.50 | 241 | 2028 | 2755 | 24.8 | 18.4 | 2834 | 34.7 |
| SD | | 0.4 | 0.04 | 8.0 | 0.58 | 69 | 183 | 380 | 15.0 | 10.2 | 361 | 4.0 |
| CV % | | 4.1 | 4.1 | 57.8 | 38.5 | 28.5 | 9.05 | 13.8 | 60.5 | 55.6 | 12.7 | 11.6 |

TABLE 4

| | No. of | Body | Actual | | | | | | | | AUC_{Inf}/D | |
|---|---|---|---|---|---|---|---|---|---|---|---|---|
| | pts used | Weight | Dosage | T_{1/2} | Tₘₐₓ | Cₘₐₓ | AUCₗₐₛₜ | AUC_{Inf} | AUC_{Extr} | MRT_{Inf} | (hr * kg * ng/ | F |
| Animal | for T_{1/2} | (kg) | (mg/kg) | (hr) | (hr) | (ng/mL) | (hr * ng/mL) | (hr * ng/mL) | (%) | (hr) | mL/mg) | (%) |
| Dog #9 | 3 | 10.8 | 0.926 | 12.2 | 0.5 | 273 | 1641 | 2105 | 22.0 | 15.0 | 2273 | 29.4 |
| (8251624) | | | | | | | | | | | | |
| Dog #10 | 3 | 11.0 | 0.909 | 15.0 | 1 | 649 | 2845 | 3712 | 23.4 | 15.5 | 4084 | 52.0 |
| (8231364) | | | | | | | | | | | | |
| Dog #11 | 3 | 11.1 | 0.901 | 12.6 | 1 | 512 | 3882 | 5047 | 23.1 | 15.7 | 5602 | 71.6 |
| (8246116) | | | | | | | | | | | | |
| Dog #12 | 3 | 12.0 | 0.833 | 12.5 | 2 | 334 | 1919 | 2440 | 21.3 | 14.8 | 2929 | 38.3 |
| (8231780) | | | | | | | | | | | | |
| Mean | | 11.2 | 0.892 | 13.1 | 1.13 | 442 | 2572 | 3326 | 22.5 | 15.3 | 3722 | 47.8 |
| SD | | 0.5 | 0.041 | 1.3 | 0.63 | 171 | 1014 | 1340 | 0.9 | 0.4 | 1460 | 18.4 |
| CV % | | 4.7 | 4.6 | 9.8 | 55.9 | 38.7 | 39.4 | 40.3 | 4.2 | 2.9 | 39.2 | 38.4 |

TABLE 5

| | No. of | Body | Actual | | | | | | | | AUC_{Inf}/D | |
|---|---|---|---|---|---|---|---|---|---|---|---|---|
| | pts used | Weight | Dosage | T_{1/2} | Tₘₐₓ | Cₘₐₓ | AUCₗₐₛₜ | AUC_{Inf} | AUC_{Extr} | MRT_{Inf} | (hr * kg * ng/ | F |
| Animal | for T_{1/2} | (kg) | (mg/kg) | (hr) | (hr) | (ng/mL) | (hr * ng/mL)) | (hr * ng/mL) | (%) | (hr) | mL/mg) | (%) |
| Dog #13 | 3 | 11.6 | 2.59 | 9.98 | 2 | 302 | 3281 | 4072 | 19.4 | 14.4 | 1572 | 21.1 |
| (8102157) | | | | | | | | | | | | |
| Dog #14 | 3 | 11.5 | 2.61 | 12.3 | 0.5 | 993 | 5884 | 7783 | 24.4 | 16.2 | 2982 | 37.5 |
| (8252809) | | | | | | | | | | | | |
| Dog #15 | 3 | 11.9 | 2.52 | 17.8 | 1 | 1440 | 8769 | 12588 | 30.3 | 20.0 | 4995 | 57.8 |
| (8010083) | | | | | | | | | | | | |
| Dog #16 | 3 | 11.0 | 2.73 | 13.6 | 0.5 | 954 | 6285 | 8620 | 27.1 | 18.1 | 3157 | 38.3 |
| (8996726) | | | | | | | | | | | | |
| Mean | | 11.5 | 2.61 | 13.4 | 1.00 | 922 | 6055 | 8266 | 25.3 | 17.2 | 3177 | 38.6 |
| SD | | 0.4 | 0.09 | 3.3 | 0.71 | 469 | 2247 | 3494 | 4.6 | 2.4 | 1405 | 15.0 |
| CV % | | 3.3 | 3.3 | 24.3 | 70.7 | 50.8 | 37.1 | 42.3 | 18.2 | 14.0 | 44.2 | 38.8 |

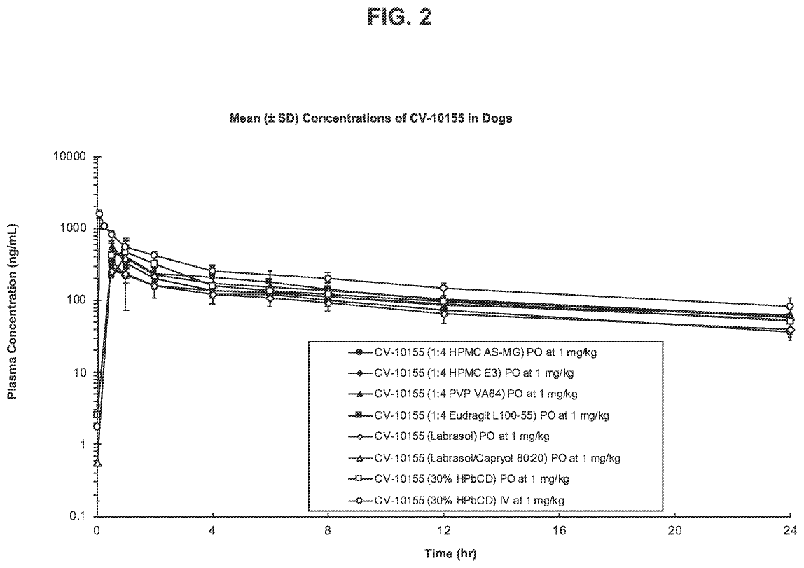

FIG. 2 is a graph of average plasma concentration of CV-10155 at various time points following oral administration to dogs.

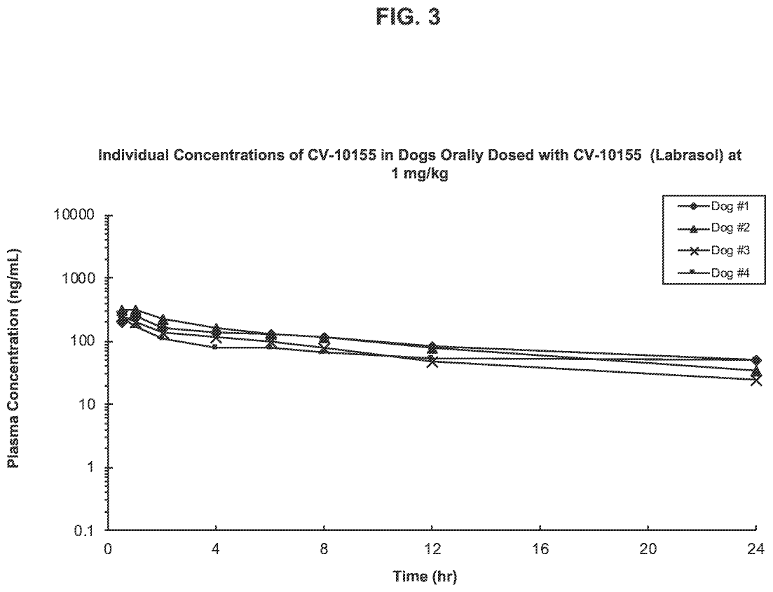

FIG. 3 is a graph of plasma concentration of CV-10155 in individual dogs at various time points following oral administration of 1 mg/kg CV-10155 in a formulation containing labrasol.

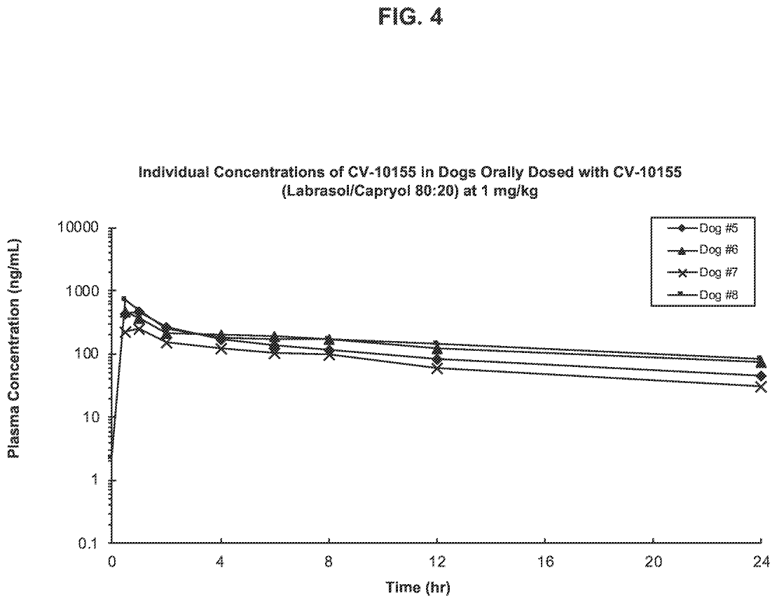

FIG. 4 is a graph of plasma concentration of CV-10155 in individual dogs at various time points following oral administration of 1 mg/kg CV-10155 in a formulation containing labrasol/capryol 80:20.

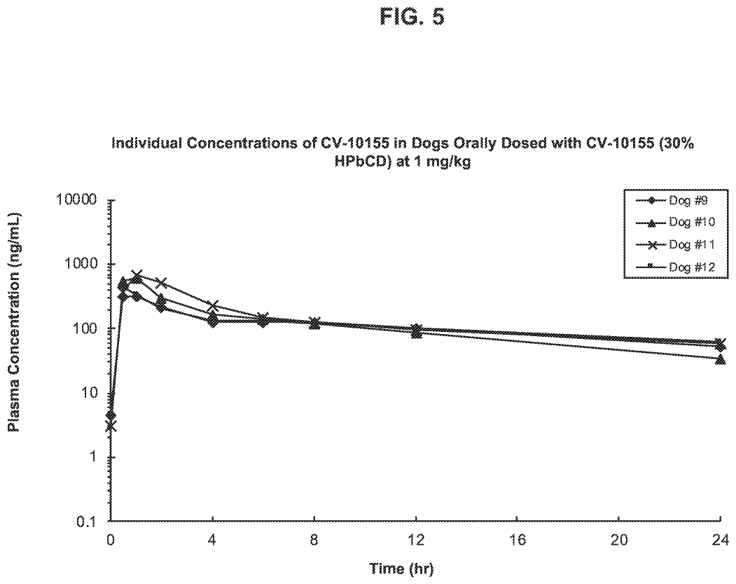

FIG. 5 is a graph of plasma concentration of CV-10155 in individual dogs at various time points following oral administration of 1 mg/kg CV-10155 in a formulation containing 30% 2-hydroxypropyl-beta-cyclodextrin (HPbCD).

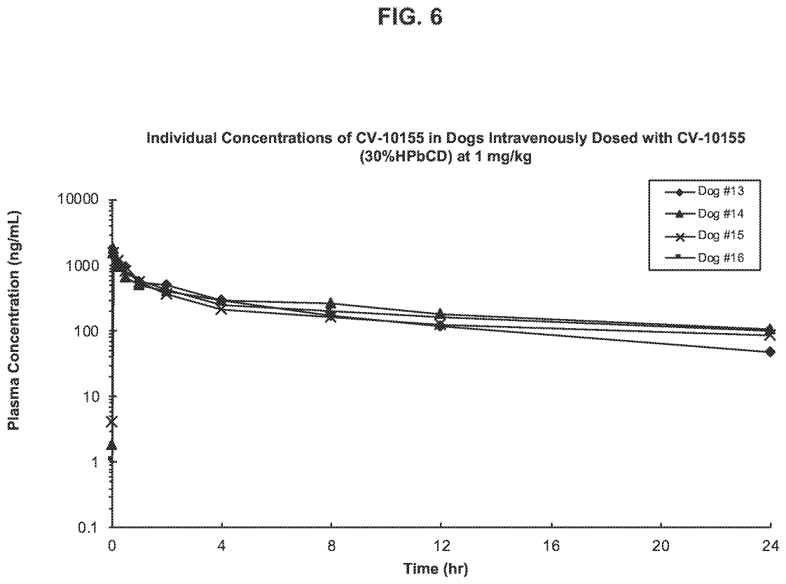

FIG. 6 is a graph of plasma concentration of CV-10155 in individual dogs at various time points following intravenous administration of 1 mg/kg CV-10155 in a formulation containing 30% HPbCD.

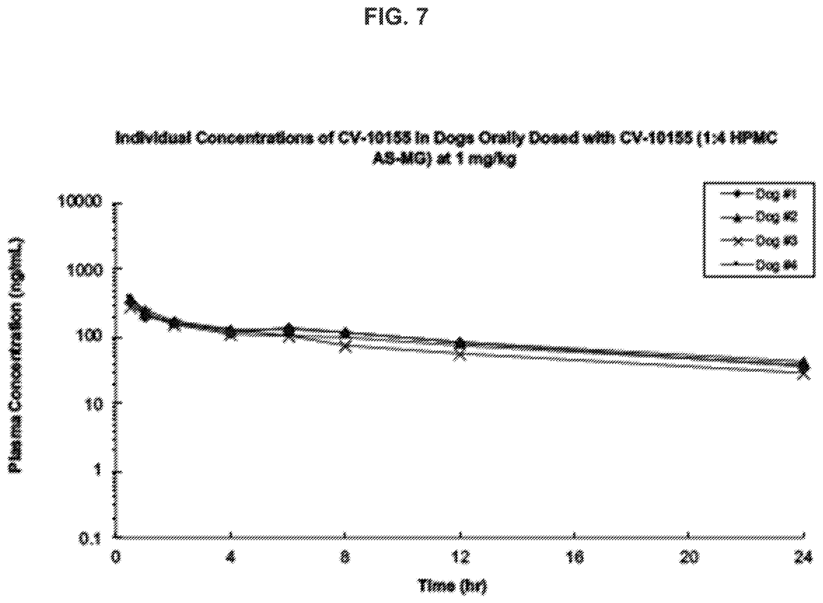

FIG. 7 is a graph of plasma concentration of CV-10155 in individual dogs at various time points following oral administration of 1 mg/kg CV-10155 in a formulation containing 1:4 HPMC-AS-MG.

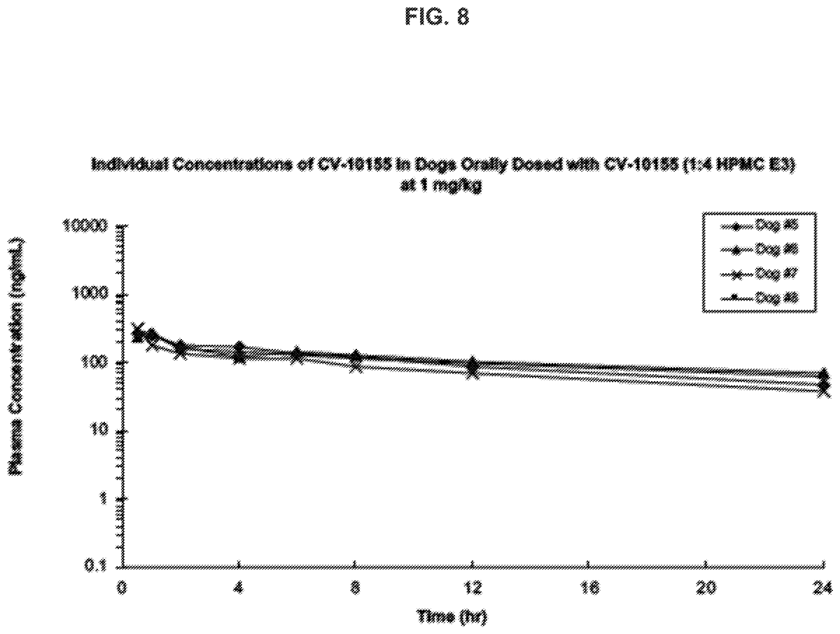

FIG. 8 is a graph of plasma concentration of CV-10155 in individual dogs at various time points following oral administration of 1 mg/kg CV-10155 in a formulation containing 1:4 HPMC-E3.

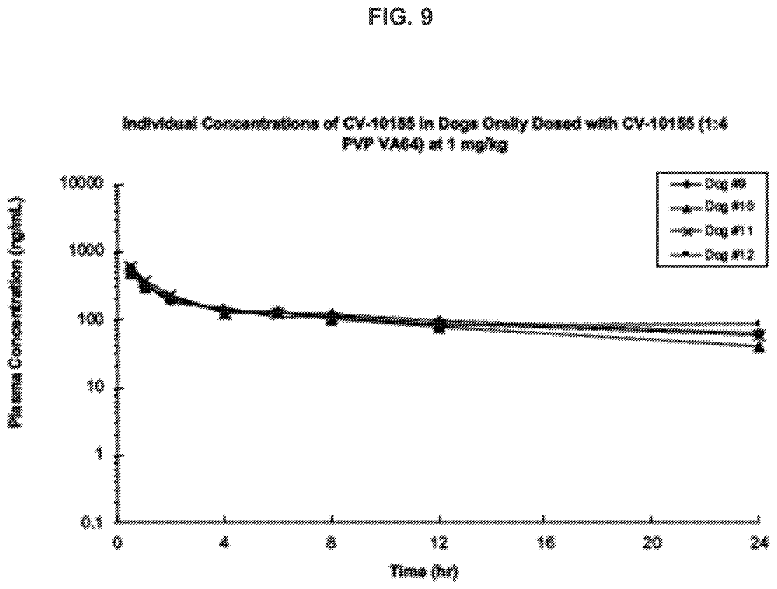

FIG. 9 is a graph of plasma concentration of CV-10155 in individual dogs at various time points following oral administration of 1 mg/kg CV-10155 in a formulation containing 1:4 PVP VA64.

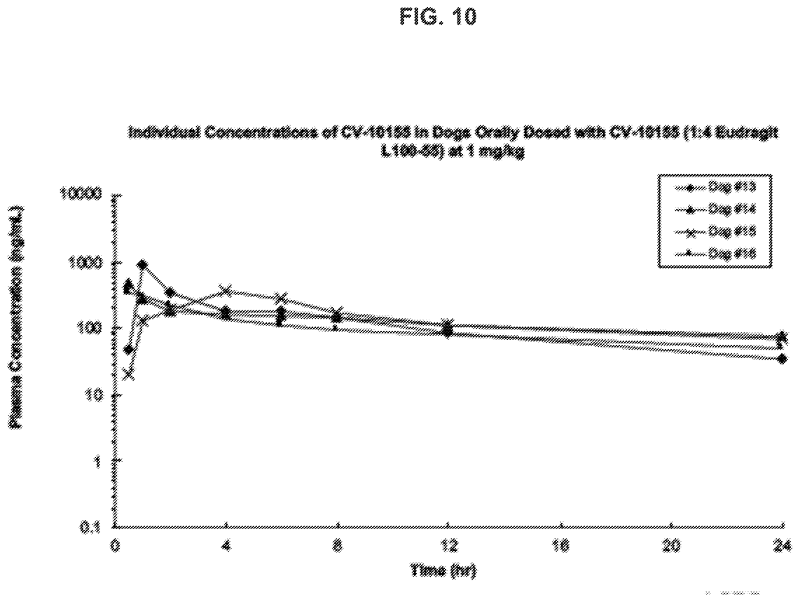

FIG. 10 is a graph of plasma concentration of CV-10155 in individual dogs at various time points following oral administration of 1 mg/kg CV-10155 in a formulation containing 1:4 Eudragit L100-55.

Data from studies of pharmacokinetics of CV-10155 in dogs are summarized in Table 6.

TABLE 6

| | Dose in | | | | | | | | | | |
|---|---|---|---|---|---|---|---|---|---|---|---|
| | Dogs | | T_{1/2} (hr) | Tₘₐₓ | Tₘₐₓ (hr) | Cₘₐₓ | Cₘₐₓ (ng/mL) | AUC_{Inf}/ | AUC_{Inf}/D | F | F (%) |
| Formulation | (mg/kg) | T_{1/2} (hr) | CV % | (hr) | CV % | (ng/mL) | CV % | D | CV % | (%) | CV % |
| CV-10155 (30% | 1 | 13.93 | 31.1 | N/A | N/A | 1732 | 16.4 | 7789 | 13.02 | 100 | 13.02 |
| HPbCD) IV | | | | | | | | | | | |
| administration | | | | | | | | | | | |
| CV-10155 SDD-1 (1:4 | 1 | 11.38 | 10.9 | 0.5 | 0 | 345 | 15.4 | 2800 | 12.1 | 36.4 | 11.8 |
| HPMC AS-MG) PO | | | | | | | | | | | |
| administration | | | | | | | | | | | |
| CV-10155 SDD-2 (1:4 | 1 | 15.87 | 21.5 | 0.625 | 40 | 280 | 8.9 | 3758 | 22.9 | 38.4 | 15.6 |
| HPMC E3) PO | | | | | | | | | | | |
| administration | | | | | | | | | | | |
| CV-10155 SDD-3 (1:4 | 1 | 17.78 | 26.4 | 0.5 | 0 | 568 | 9.6 | 4421 | 23.7 | 45.6 | 6.3 |
| PVP VA64) PO | | | | | | | | | | | |
| administration | | | | | | | | | | | |
| CV-10155 SDD-4 (1:4 | 1 | 13.85 | 32.5 | 1.5 | 112.2 | 517 | 48.8 | 4313 | 17.1 | 51.6 | 14.1 |
| Eudragit L100-55) PO | | | | | | | | | | | |
| administration | | | | | | | | | | | |
| CV-10155 (Labrosol) | 1 | 18.93 | 81.3 | 0.625 | 40 | 266 | 12.7 | 3268 | 32.9 | 34.1 | 21.8 |
| PO administration | | | | | | | | | | | |
| CV-10155 | 1 | 13.31 | 20.4 | 0.75 | 38.5 | 477 | 41.1 | 4333 | 37.3 | 51.7 | 29.1 |
| (Labrosol/Capryol | | | | | | | | | | | |
| 80:20) PO | | | | | | | | | | | |
| administration | | | | | | | | | | | |
| CV-10155 (30% | 1 | 13.62 | 27.3 | 0.75 | 38.5 | 510 | 31.7 | 4175 | 15.6 | 51.5 | 14.0 |
| HPbCD) PO | | | | | | | | | | | |
| administration | | | | | | | | | | | |

### Example 2

The potency of the neurosteroids CV-10155, Sage-217, ganaxolone, and Praxis-114 on GABA_{A} receptors in preclinical studies was compared. Results are summarized in Table 7.

TABLE 7

| EC50 - potency (Emax - efficacy: | | | | Praxis-114 (S1 SEC |
|---|---|---|---|---|
| % of potentiation) | ETX-155* | Sage-217* | Ganaxolone* | report) |
| α1β2γ2 Synaptic receptors | 207 (586%) | 189 (1000%) | 90 (745%) | 2241 (~1700%) |
| α4β3δ Extra synaptic receptors | 165 (1530%) | 148 (3080%) | 213 (3320%) | 353 (~900%) |

*Data obtained by Synchropatch electrophysiology

The data show that CV-10155 is a dual potent synaptic and extra synaptic GABA_{A} positive allosteric modulator (PAM), with higher efficacy at extra synaptic channels. The dual potency of CV-10155 at synaptic and extra synaptic channels is similar to that of Sage-217 but different from Praxis-114, which is ˜7-fold more potent at extrasynaptic receptors. CV-10155 has 3-fold higher efficiency at extra synaptic vs. synaptic channels.

The effects of the neurosteroids CV-10155, Sage-217, and ganaxolone on various animal behaviors in preclinical studies were compared. Results are summarized in Table 8.

TABLE 8

| | ETX-155 | SAGE-217 | Ganaxolone |
|---|---|---|---|
| | (mg/kg) | (mg/kg) | (mg/kg) |
| MES - Seizure | 1 - 3 - - 10* | | 1 - 3 - 10 |
| PTZ - Seizure | l* - 3* - - 10* | | 1 - 3 - 10 |
| GAERS - Absence seizure | | | |
| Marble burying - | 1 - 3 - 6* | | |
| Depression/Anxiety | | | |
| Forced swim test - Depression | 1 - 3* - - 10* | 1 - 3* | |
| Elevated Plus Maze - Anxiety | 1 - 3 - 6* - 10* | 1 - 3* - 6* | |
| Social interaction - | 1* - 3* - 6* | | |
| Anxiety/Depression | | | |
| EEG/Sleep | 1 - 3* - 6* - 10* | | |
| Sleep | 1 - 3* - 6* - 10* | | |

*Significantly efficacious dose.

The results show that CV-10155 is efficacious at 3mg/kg in a broad range of preclinical models of depression, anxiety, seizure, and EEG. Overall, CV-10155 show efficacy in models of depression, anxiety, and seizure. The minimal effective dose (MED) is 1mg/kg, robust efficacy was observed from 3mg/kg onwards in most models.

The pharmacokinetic parameters of CV-10155 in preclinical studies on rodent models of seizure, anxiety, depression and EEG biomarker were analyzed. The results are summarized in Table 9.

TABLE 9

| | Minimal effective dose | | Effective Dose | | |
|---|---|---|---|---|---|
| ETX-155 | 1 mg/kg | 2 mg/kg | 3 mg/kg | 5 mg/kg | 10 mg/kg |
| Cₘₐₓ | 84.4-128 | 224 | 270-440 | 499 | 1165 |
| Tₘₐₓ | 1.5-2 | 1.7 | 1.25-2.50 | 0.83 | 1.25-2.25 |
| AUC_{0-inf} | 508-1301 | 1319 | 1378-3899 | 2610 | 6848-14667 |
| ½ life | 3.66-6.82 | nd | 2.84-4.96 | 4.19 | 3.23-4.80 |
| Oral BA | 58.6-92.4 | 47.2 | 53-92.3 | 58.1 | 79.7-100 |

1, 3 and 10 mg/kg data from study including male and female subjects
2 and 5 mg/kg data from study including only male subjects

The results show that preclinical minimal efficacious dose is 1 mg/kg, and robust efficacy observed from 3 mg/kg onwards across several rodent models of seizure, anxiety, depression and EEG biomarker.

### Example 3

The pharmacokinetic properties of CV-10155 in humans were analyzed. Subjects were given a single oral dose of CV-10155, and plasma concentrations were measured at various time points. Various dosages were given to fasting subjects, and 30 mg dose was given to a subjects in a fed state.

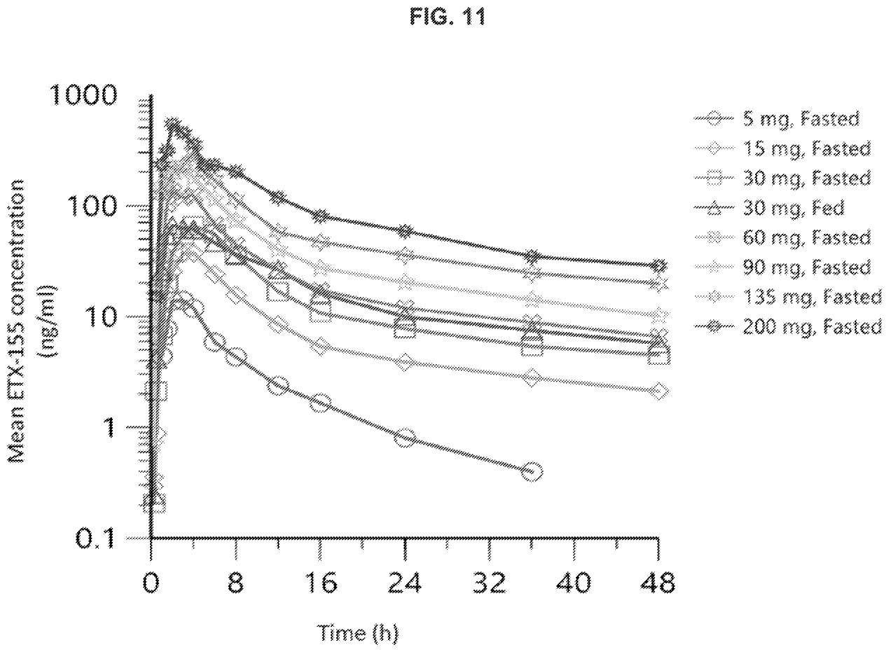

FIG. 11 is a graph of the average plasma concentration of CV-10155 following oral administration to humans. Purple circles represent 5 mg, fasted; green diamonds represent 15 mg, fasted; red squares represent 30 mg fasted; blue triangles represent 30 mg, fed; purple crosses represent 60 mg, fasted; teal 5-pointed stars represent 90 mg, fasted; olive 6-pointed stars represent 135 mg, fasted; and brown 10-pointed stars represent 200 mg fasted.

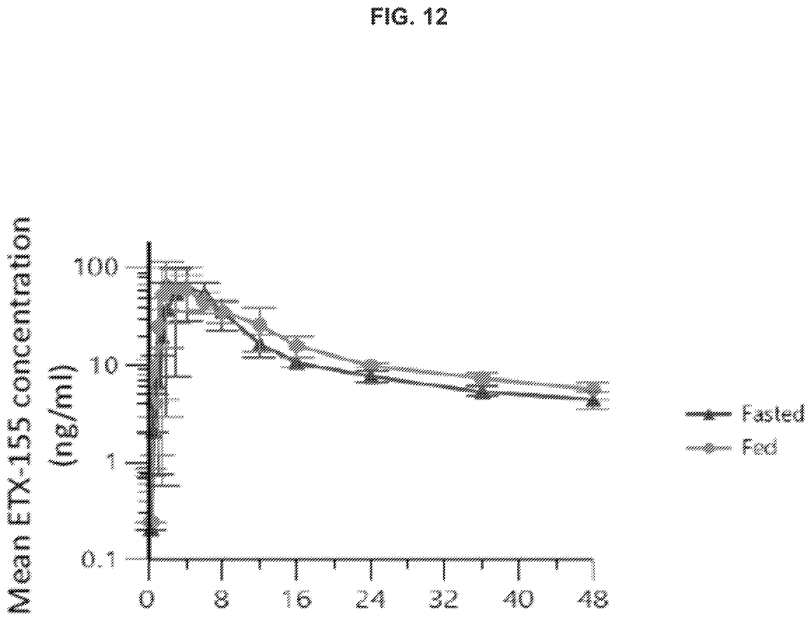

FIG. 12 is a graph of the average plasma concentration of CV-10155 following oral administration of a 30 mg dose to humans. Blue triangles represent fasted; and red circles represent fed.

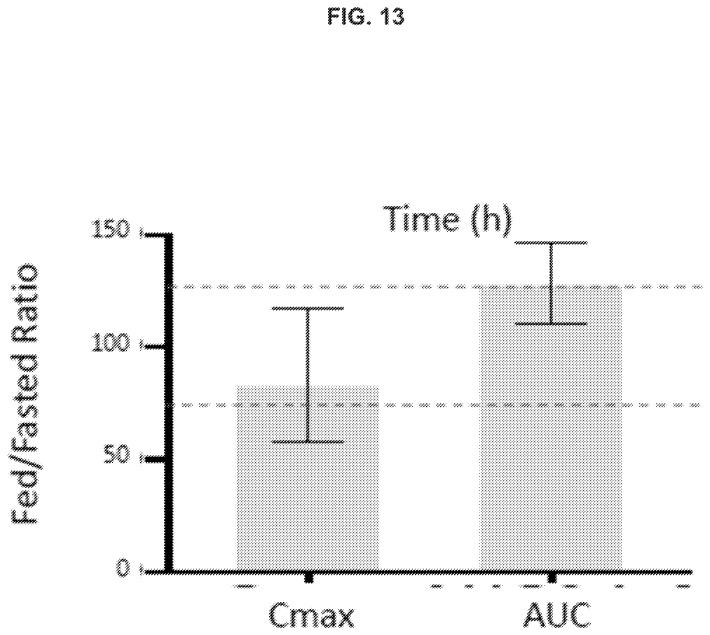

FIG. 13 is a graph showing the ratios of Cₘₐₓ and AUC between fed and fasted subjects.

CV-10155 displays a dose-proportional increase for AUC and Cₘₐₓ across 5-200 mg dose range with small/moderate inter-subject variability (CV≤30%). The Tₘₐₓ is ˜2-4 hours, and the half-life is ˜24-26 hours. No dose-limiting adverse events were observed over the range of 5-135 mg; ataxia, tremor and tachycardia were observed at 200 mg. Dizziness and somnolence, which were mild to moderate, were the most common adverse events and were only observed at the highest doses.

Taken together, the results show that food consumption has no clinically meaningful effect on the absorption of CV-10155 following oral administration of the drug. These findings support the oral administration of CV-10155 in a dosing regimen that is agnostic regarding food consumption. In particular, the results indicate that CV-10155 is suitable for oral administration during fasting periods and need not be taken with food.

### Example 4

The effects of CV-10155 in relation to administration at different times of the day and states of feeding were analyzed in humans. Subjects were given 60 mg CV-10155 orally once per day for seven consecutive days, and plasma concentrations were measured at various time points. Doses were administered either in the morning to fasted subjects or in the evening to fed subjects.

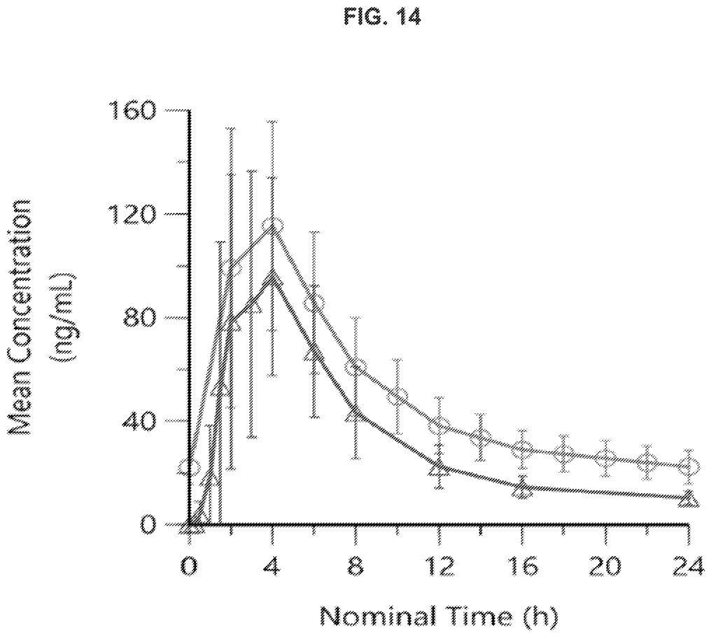

FIG. 14 is a graph of the average plasma concentration of CV-10155 following oral administration to humans. Red circles represent morning, fasted administration; and blue triangles represent evening, fed administration.

Adverse events observed following administration of a single dose of CV-10155 in the evening in a fed state are summarized on Table 10.

TABLE 10

| Subject | AE | Dosing day | Onset from dose (h) | Duration (h) | Severity |
|---|---|---|---|---|---|
| a | Somnolence | 1 | 00:45 | 10.62 | Mild |
| v | Back pain | 4 | 00:15 | 39.25 | Mild |
| l | Headache | 1 | pre-dose | 13.25 | Mild |
| o | Abdominal distension | 3 | 17:20 | 06.50 | Mild |
| p | Dizziness | 2 | 01:10 | 20.50 | Mild |
| p | Dizziness | 7 | 01:10 | 13.00 | Mild |
| p | Headache | 8 | 21:40 | 14.50 | Moderate |

A comparison of adverse events observed following administration to fasted subjects in the morning and to fed subjects in the evening is provided in Table 11.

TABLE 11

| | | | N = 9 | N = 9 |
|---|---|---|---|---|
| | | N = 3 | 60 mg, morning, | 60 mg, evening, |
| | AE | Placebo | fasted state | fed state |
| AE SOC | description | # | # | # |
| Gastrointestinal disorders | Bloating | — | — | 1 |
| General disorders and | Tiredness | — | 3 | — |
| administration site conditions | Feeling tired | — | 2 | — |
| Musculoskeletal and connective | Back pain | — | — | 1 |
| tissue disorders | | | | |
| Nervous system disorders | Sleepiness | 2 | 9 | — |
| | Headache | 2 | 2 | — |
| | Dizziness | — | 1 | 1 |
| | Feeling | — | 1 | — |
| | sleepy | | | |
| Nervous system disorders | Sleepiness | — | — | 1 |
| | Headache | — | — | 2 |
| Nervous system disorders | Sleepiness | — | — | 1 |

The results show no significant difference in absorption of CV-10155 following oral administration between subjects that received it in the morning in a fasted state and subjects that received it in the evening in a fed state. In addition, oral administration of CV-10155 in the evening to fed subjects does not produce adverse effects that interfere with sleeping.

Taken together, the results indicate that oral formulations of CV-10155 are suitable for a dosing regimen in which the drug is provided in the evening and/or prior to an extended period of sleep.

### Example 5

The pharmacokinetic properties of CV-10155 obtained from preclinical studies and from studies on humans were compared to determine comparable dosing levels between animals and humans.

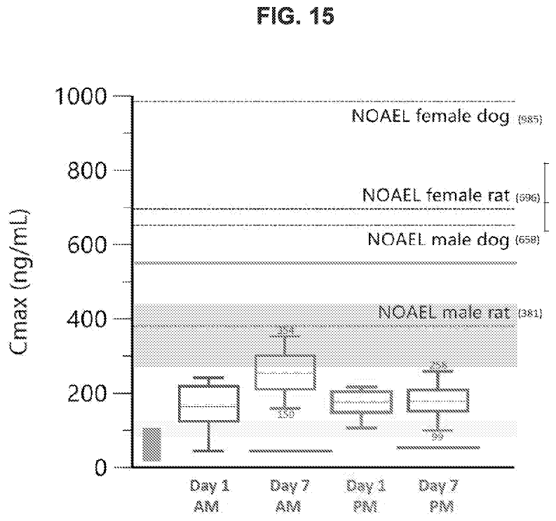

FIG. 15 is a graph of Cₘₐₓ ranges from studies on rats, dogs, and humans. Blue open box represents day 1 data from humans given oral dose in the morning in a fasted state; green open box represents day 7 data from humans given oral dose in the morning in a fasted state; red open box represents day 1 data from humans given oral dose in the evening in a fed state; purple open box represents day 7 data from humans given oral dose in the evening in a fed state; solid orange line indicates levels associated with ataxia in humans; solid dark green box indicates levels associated with robust efficacy in preclinical studies, and solid light green box indicates levels associated with the minimum effective dose in preclinical studies. The ratios of C_{max/trough} on day 7 from human studies are as follows: for 60 mg CV-10155 administered daily in the morning in a fasted state, 6.09; for 60 mg CV-10155 administered daily in the evening in a fed state, 3.47; and for 30 mg Sage-2017 administered daily in the evening in a fed state, 5.88.

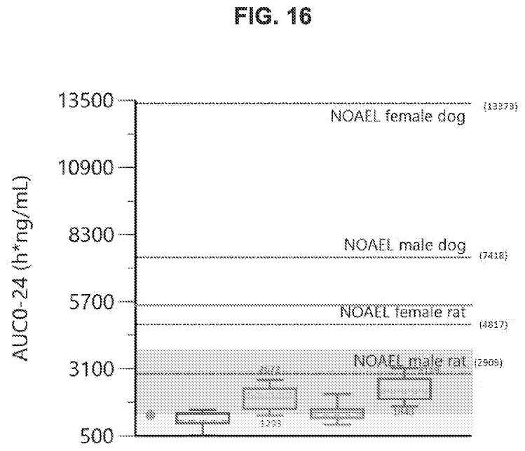

FIG. 16 is a graph of AUC₀₋₂₄ ranges from studies on rats, dogs, and humans. Blue open box represents day 1 data from humans given oral dose in the morning in a fasted state; green open box represents day 7 data from humans given oral dose in the morning in a fasted state; red open box represents day 1 data from humans given oral dose in the evening in a fed state; purple open box represents day 7 data from humans given oral dose in the evening in a fed state; solid orange line indicates levels associated with ataxia in humans; solid dark green box indicates levels associated with robust efficacy in preclinical studies, and solid light green box indicates levels associated with the minimum effective dose in preclinical studies.

Taken together, the results show that the values of pharmacokinetic parameters resulting from oral administration of 60 mg CV-10155 in humans are similar to those observed when the drug is provided in efficacious doses to dogs and rats in preclinical animal models.

### Example 6

The tolerability of the neurosteroids CV-10155, Sage-217, and Praxis-114 in human subjects were compared. The results from subjects that received 45 mg Praxis-114 in the evening, 60 mg Praxis-114 in the evening, 80 mg Praxis-114 in the evening, or 60 mg CV-10155 in the evening are provided in Table 12, and the results form subjects that received 30 mg Sage-217 or 20 mg Sage-217 are provided in Table 13.

TABLE 12

| | Praxis-114 MDD | Praxis-114 MDD | Praxis-114 MDD | ETX-155 HV |
|---|---|---|---|---|
| | 45 mg | 60 mg | 80 mg | 60 mg |
| | evening dosing | evening dosing | evening dosing | Evening dosing |
| | AEs % of subjects | AEs % of subjects | AEs % of subjects | AEs % of subjects |
| | (n = 13) | (n = 13) | (n = 7) | (n = 12) |
| Somnolence | 15.4% | 53.8% | 42.9% | 8.3% |
| Fatigue | 23.1% | — | — | — |
| Headache | 53.8% | 46.2% | 42.9% | 8.3% |
| Dizziness | — | 30.8% | 57.1% | 16.7% |
| Feeling drunk | — | 23.1% | 28.6% | — |

TABLE 13

| | Sage-217 30 mg | Sage-217 20 mg | Placebo |
|---|---|---|---|
| | (n-192) | (n-188) | (n = 190) |
| Any - n (%) | 104 (54.2) | 94 (50.0) | 93 (48.9) |
| Headache | 12 (6.3) | 21 (11.2) | 14 (7.4) |
| Dizziness | 11 (5.7) | 14 (7.4) | 7 (3.7) |
| Somnolence | 13 (6.8) | 11 (5.9) | 8 (4.2) |
| Fatigue | 13 (6.8) | 3 (1.6) | 5 (2.6) |
| Diarrhea | 12 (6.3) | 11 (5.9) | 10 (5.3) |
| Sedation | 9 (4.7) | 11 (5.9) | 6 (3.2) |
| Nausea | 7 (3.6) | 10 (5.3) | 9 (4.7) |

The results show that CNS adverse events, such as somnolence, fatigue, dizziness, are common to all three compounds. Ataxia is the dose-limiting adverse event for CV-10155. The data suggest that at 60 mg dosed in the evening, CV-10155 has a favorable tolerability profile compared to PRAX-114 and one comparable to that of Sage-217.

### Example 7

The effect of CV-10155 on various sleep states was analyzed in rats. Rats were given various doses of CV-10155, and sleep states were analyzed by electroencephalogram (EEG).

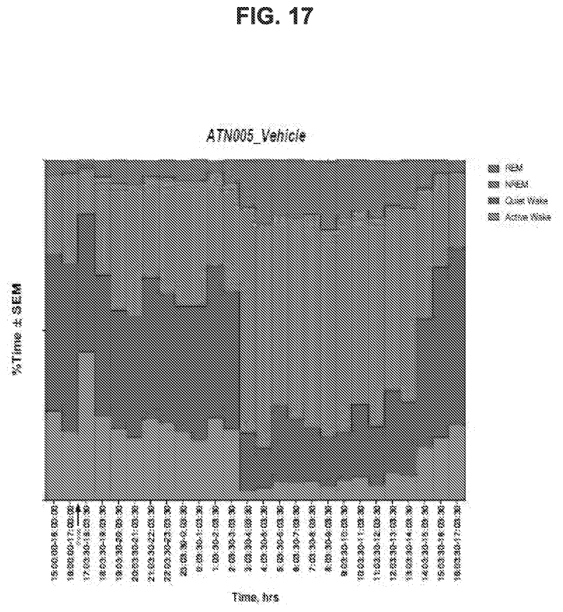

FIG. 17 is a hypnogram showing the percentage of time spent in different sleep states by rats given drug vehicle. Purple bars represent REM sleep; green bars represent non-REM sleep; blue bars represent quiet waking; and red bars represent active waking.

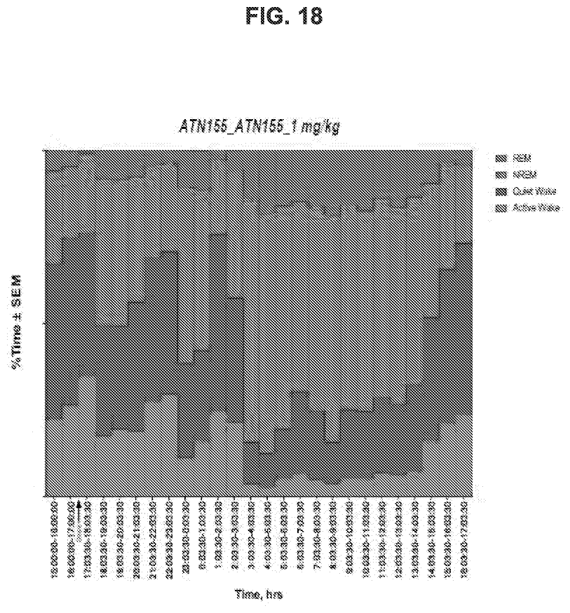

FIG. 18 is a hypnogram showing the percentage of time spent in different sleep states by rats given 1 mg/kg CV-10155. Purple bars represent REM sleep; green bars represent non-REM sleep; blue bars represent quiet waking; and red bars represent active waking.

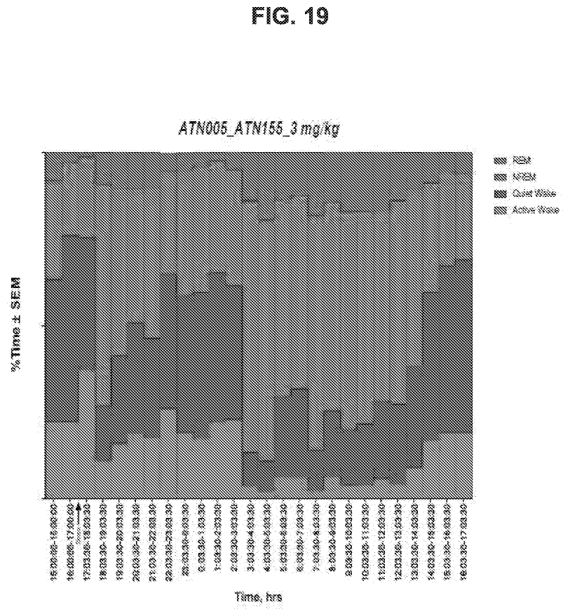

FIG. 19 is a hypnogram showing the percentage of time spent in different sleep states by rats given 3 mg/kg CV-10155. Purple bars represent REM sleep; green bars represent non-REM sleep; blue bars represent quiet waking; and red bars represent active waking.

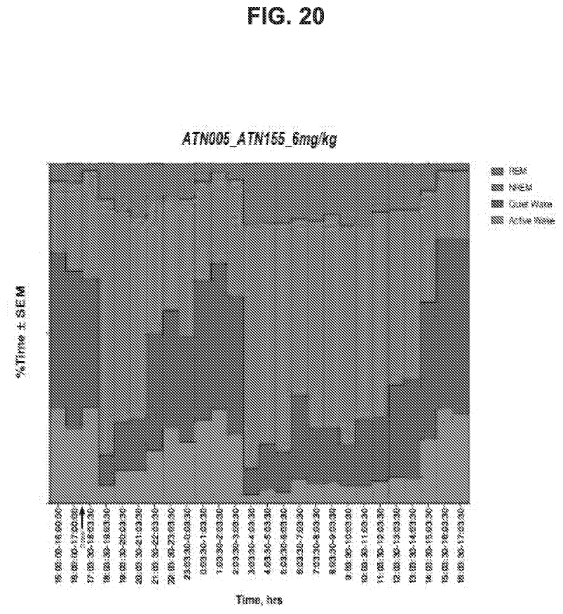

FIG. 20 is a hypnogram showing the percentage of time spent in different sleep states by rats given 6 mg/kg CV-10155. Purple bars represent REM sleep; green bars represent non-REM sleep; blue bars represent quiet waking; and red bars represent active waking.

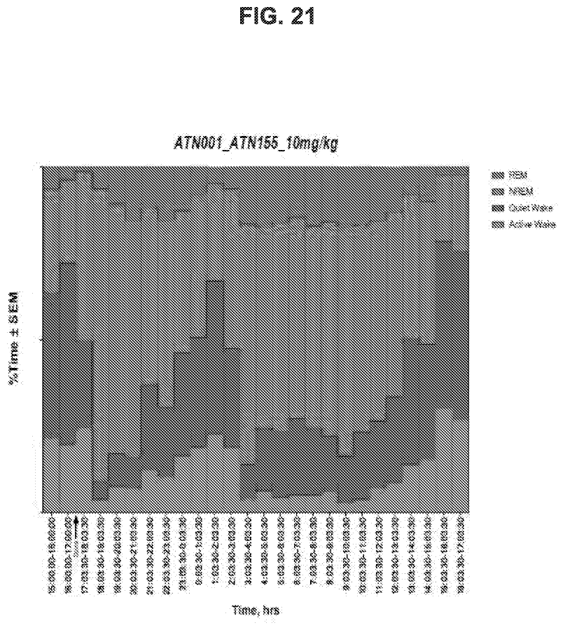

FIG. 21 is a hypnogram showing the percentage of time spent in different sleep states by rats given 10 mg/kg CV-10155. Purple bars represent REM sleep; green bars represent non-REM sleep; blue bars represent quiet waking; and red bars represent active waking.

The results show that CV-10155 displays dose-dependent target engagement within well-tolerated and efficacious dose ranges. In addition, CV-10155 produces an increase in non-REM sleep at doses of 3 mg/kg and higher.

These findings indicate that CV-10155 improves sleep quality in animal models and suggest that CV-10155 may be useful to treat sleep disorders in human.

### Example 8

The pharmacokinetic properties of CV-10155 and Sage-217 in humans were compared. The day 7 from subjects given either 60 mg CV-10155 in the morning in a fasted state or 30 mg Sage-217 were analyzed. Results from multiple ascending dose studies are shown in Table 14, and results from single ascending dose studies are shown in Table 15.

TABLE 14

| | Cₘₐₓ | Cₘᵢₙ | | | | AUC_{0-inf} | Racc | Racc |
|---|---|---|---|---|---|---|---|---|
| MAD | (ng/ml) | (ng/ml) | Ratio | Tₘₐₓ (h) | T_{1/2} (h) | (h * ng/ml) | Cₘₐₓ | AUC |
| ETX-155 60 mg | 252.56 | 41.5 | 6.07 | 3 | 34.48 | 2899.9 | 1.78 | 1.84 |
| day 7 MAD | | | | | | | | |
| Morning dosing | | | | | | | | |
| SAGE-217 30 mg | 115.2 | 12.46 | 9.24 | 1 | 15.27 | 833.2 | 0.943 | 1.298 |
| day 7 MAD (MTD) | | | | | | | | |
| Morning dosing | | | | | | | | |

TABLE 15

| SAD | Cₘₐₓ (ng/ml) | Tₘₐₓ (h) | T_{1/2} (h) | AUC_{0-inf} (h*ng/ml) |
|---|---|---|---|---|
| ETX-155 135 mg SAD MTD | 332.8 | 2.83 | 25.0 | 3661 |
| SAGE-217 55 mg SAD MTD | 149.9 | 1.24 | 17.02 | 1633 |

Taken together, the results show that administration of 60 mg doses of CV-10155 achieves higher exposure levels in humans than does administration of 30 mg doses of Sage-217.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method for treating a GABA_{A} disorder in a subject, the method comprising providing to a subject having a GABA_{A} disorder a dose of a composition comprising a compound of Formula (I): 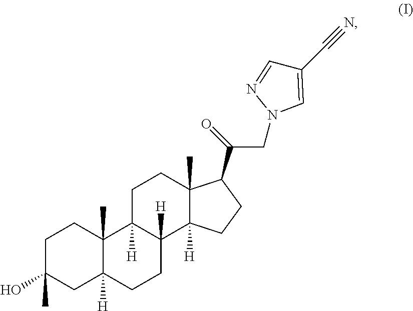
in an evening of the subject's local time.

2. The method of claim 1, wherein the composition comprises an isomerically pure form of the compound of Formula (I).

3. The method of claim 2, wherein the composition comprises the compound of Formula (I) at an isomeric purity of at least 98% by weight.

4. The method of claim 1, wherein the compound of Formula (I) is provided in a therapeutically effective amount to preferentially potentiate an α4β3δ GABA_{A} receptor as compared to an α1β2γ2 GABA_{A} receptor.

5. The method of claim 4, wherein an EC50 of the compound of Formula (I) for an α4β3δ GABA_{A} receptor is less than 50% of an EC₅₀ of the compound of Formula (I) for an α1β2γ2 GABA_{A} receptor.

6. The method of claim 4, wherein an EC₅₀ of the compound of Formula (I) for an α4β3δ GABA_{A} receptor is less than 20% of an EC₅₀ of the compound of Formula (I) for an α1β2γ2 GABA_{A} receptor.

7. The method of claim 4, wherein an EC50 of the compound of Formula (I) for an α4β3δ GABA_{A} receptor is less than 500 nM.

8. The method of claim 1, wherein the GABA_{A} disorder is selected from the group consisting of acute pain, an addictive disorder, Alzheimer's disease, Angelman's syndrome, anti-social personality disorder, an anxiety disorder, attention deficit hyperactivity disorder (ADHD), an attention disorder, an auditory disorder, autism, an autism spectrum disorder, bipolar disorder, chronic pain, a cognitive disorder, a compulsive disorder, a convulsive disorder, dementia, depression, dysthymia, an epileptic disorder, essential tremor, epileptogenesis, fragile X syndrome, generalized anxiety disorder (GAD), Huntington's disease, injury related pain syndrome, insomnia, ischemia, Lewis body type dementia, a memory disorder, migraines, a mood disorder, movement disorder, a neurodegenerative disease, neuropathic pain, an obsessive compulsive disorder, pain, a panic disorder, Parkinson's disease, a personality disorder, posttraumatic stress disorder (PTSD), psychosis, Rett syndrome, a schizoaffective disorder, schizophrenia, a schizophrenia spectrum disorder, a seizure disorder, a sleep disorder, social anxiety disorder, status epilepticus, stress, stroke, tinnitus, traumatic brain injury (TBI), vascular disease, vascular malformation, vascular type dementia movement disorder, Wilson's disease, and withdrawal syndrome.

9. The method of claim 8, wherein the GABA_{A} disorder is an anxiety disorder, depression, or a seizure disorder.

10. The method of claim 8, wherein the GABA_{A} disorder is insomnia or a sleep disorder.

11. A method for treating a GABA_{A} disorder in a subject, the method comprising providing to a subject having a GABA_{A} disorder a dose of a composition comprising a compound of Formula (I): 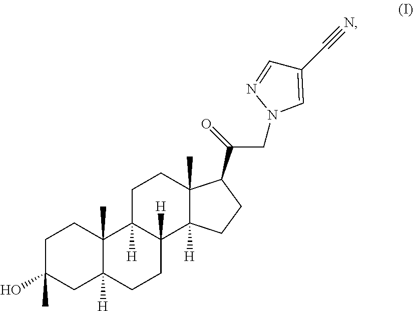
at a dosing time that is less than 6 hours before a time when the subject goes to sleep.

12. The method of claim 11, wherein the dosing time is less than 4 hours before the time when the subject goes to sleep.

13. The method of claim 11, wherein the dosing time is less than 2 hours before the time when the subject goes to sleep.

14. The method of claim 11, wherein the composition comprises an isomerically pure form of the compound of Formula (I).

15. The method of claim 14, wherein the composition comprises the compound of Formula (I) at an isomeric purity of at least 98% by weight.

16. The method of claim 11, wherein the compound of Formula (I) is provided in a therapeutically effective amount to preferentially potentiate an α4β3δ GABA_{A} receptor as compared to an α1β2γ2 GABA_{A} receptor.

17. The method of claim 16, wherein an EC₅₀ of the compound of Formula (I) for an α4β3δ GABA_{A} receptor is less than 50% of an EC₅₀ of the compound of Formula (I) for an α1β2γ2 GABA_{A} receptor.

18. The method of claim 11, wherein the GABA_{A} disorder is selected from the group consisting of acute pain, an addictive disorder, Alzheimer's disease, Angelman's syndrome, anti-social personality disorder, an anxiety disorder, attention deficit hyperactivity disorder (ADHD), an attention disorder, an auditory disorder, autism, an autism spectrum disorder, bipolar disorder, chronic pain, a cognitive disorder, a compulsive disorder, a convulsive disorder, dementia, depression, dysthymia, an epileptic disorder, essential tremor, epileptogenesis, fragile X syndrome, generalized anxiety disorder (GAD), Huntington's disease, injury related pain syndrome, insomnia, ischemia, Lewis body type dementia, a memory disorder, migraines, a mood disorder, movement disorder, a neurodegenerative disease, neuropathic pain, an obsessive compulsive disorder, pain, a panic disorder, Parkinson's disease, a personality disorder, posttraumatic stress disorder (PTSD), psychosis, Rett syndrome, a schizoaffective disorder, schizophrenia, a schizophrenia spectrum disorder, a seizure disorder, a sleep disorder, social anxiety disorder, status epilepticus, stress, stroke, tinnitus, traumatic brain injury (TBI), vascular disease, vascular malformation, vascular type dementia movement disorder, Wilson's disease, and withdrawal syndrome.

19. The method of claim 18, wherein the GABA_{A} disorder is an anxiety disorder, depression, or a seizure disorder.

20. The method of claim 18, wherein the GABA_{A} disorder is insomnia or a sleep disorder.
